(19) United States
(12) Patent Application Publication
(10) Pub. No.: US 2008/0171050 A1
(43) Pub. Date: Jul. 17, 2008

(54) TREATMENT OF HYPERPROLIFERATIVE DISEASES WITH EPIDERMAL GROWTH FACTOR RECEPTOR ANTAGONISTS

(75) Inventor: Thomas Teufel, Cape Coral, FL (US)

Correspondence Address: LUCAS & MERCANTI, LLP, 475 PARK AVENUE SOUTH, 15TH FLOOR, NEW YORK, NY, 10016, US

(73) Assignee: IMCLONE SYSTEMS INC., New York, NY (US)

(21) Appl. No.: 11/948,228

(22) Filed: Nov. 30, 2007

Related U.S. Application Data
(63) Continuation of application No. 09/635,974, filed on Aug. 9, 2000, now abandoned.

(51) Int. Cl. A61K 39/395; A61P 17/06

(52) U.S. Cl. 424/141.1; 424/130.1; 424/133.1

(57) ABSTRACT

A method is provided for treating a mammal with hyperproliferative disease stimulated by a ligand of epidermal growth factor receptor. The method includes administering an effective amount of an antagonist of a member of the EGF family of receptors. The method also includes administering an EGFR antagonist in combination with a chemotherapeutic agent and/or phototherapy.

### CROSS-REFERENCE

This application is a continuation of application Ser. No. 09/635,974, filed Aug. 9, 2000, the entire disclosure of which is incorporated by reference.

### BACKGROUND OF THE INVENTION

Normal cells proliferate by the highly controlled activation of growth factor receptors by their respective ligands. Examples of such receptors are the growth factor receptor tyrosine kinases.

Members of the epidermal growth factor (EGF) receptor family are particularly important growth factor receptor tyrosine kinases associated with both normal and excessive proliferation of epidermal cells. The first member of the EGF receptor family to be discovered was a glycoprotein having an apparent molecular weight of approximately 165 kD. This glycoprotein, which was described by Mendelsohn et al. in U.S. Pat. No. 4,943,533, is known as the EGF receptor (EGFR) and also as human EGF receptor-1 (HER1).

Hyperproliferative disease is a condition caused by the excessive growth of cells. Cells associated with hyperproliferative disease generally proliferate by the activation of growth factor receptors that lose the careful control of normal proliferation. The loss of control can be caused by numerous factors, such as the overexpression of growth factors and/or receptors, and autonomous activation of biochemical pathways regulated by growth factors.

An example of hyperproliferative disease is psoriasis. Psoriasis is a non-contagious skin disorder that most commonly appears as inflamed swollen skin lesions covered with silvery white scale. The actual cause of psoriasis is not known.

Treatments of psoriasis traditionally include various forms and combinations of topical and systemic chemotherapeutic agents. However, many of the chemotherapeutic agents conventionally used either pose the risk of serious side effects or have limited effectiveness.

For example, topical steroids account for 90% of the psoriasis market in the United States. The topical steroids currently used, however, have many side effects.

Systemic chemotherapeutic agents are also used in treating psoriasis. Potential side effects of systemic drugs include nausea, fatigue, loss of appetite, mouth sores, birth defects to developing fetuses, reduction in efficiency of the kidneys.

New types of chemotherapeutic agents that inhibit or reduce EGFR activity have been suggested as being useful in treating psoriasis. Such molecules include the tyrphostins described by Dvir, et al., J. Cell Biol., 113:857-865 (1991); the quinazoline compounds described in U.S. Pat. No. 6,004,967; the styryl substituted heteroaryl compounds disclosed in U.S. Pat. No. 5,656,655; the bis mono and/or bicyclic aryl, heteroaryl, carbocyclic, and heterocarbocyclic compounds disclosed in U.S. Pat. No. 5,646,153; the tricyclic pyrimidine compounds disclosed in U.S. Pat. No. 5,679,683; or the heteroarylethenediylaryl compounds disclosed in U.S. Pat. No. 5,196,446. These chemotherapeutic agents have not been proven effective.

The use of the anti-IL-8 human monoclonal antibody ABX-IL8 has also been disclosed for treating psoriasis. See Abgenix, Inc., “Abgenix Initiates Phase II Clinical Trial With ABX-IL8 in Psoriasis,” Company Press Release, Apr. 3, 2000. ABX-IL8 targets Interleukin-8, which is a cytokine that can cause inflammation.

Other investigations have uncovered interesting facts that may lead to new approaches to treating psoriasis. For example, it has also been demonstrated that expression of the gene Bcl-X_{L} has an anti-apoptotic effect, and that inhibition of EGFR tyrosine kinase activity with monoclonal antibody 425 downregulates Bcl-X_{L} in normal keratinocytes in culture. See Jost et al., “A Central Role of Bcl-X_{L} in the Regulation of Keratinocyte Survival by Autocrine EGFR Ligands,” J. of Invest. Derm., 112:443)-449 (1999). Similarly, Varani et al., “Human Psoriatic Skin in Organ Culture: Comparison with Normal Skin Exposed to Exogenous Growth Factors and Effects of an Antibody to the EGF Receptor,” Pathology, 66:253-259 (1998) disclose that histological features of psoriatic tissue were partially ameliorated in vitro when maintained in the presence of an antibody to EGFR. The authors note, however, that “it is difficult to extrapolate from in vitro findings to what might be occurring in vivo.” Id. p. 258.

Phototherapy has also been used to treat psoriasis. However, phototherapy treatment also has side effects and has demonstrated a limited effectiveness.

Therefore, because current treatments for hyperproliferative disease have proven to be insufficient, there is a need for new types of drugs for treating hyperproliferative disease.

### SUMMARY OF THE INVENTION

This and other objectives, as will be apparent to those having ordinary skill in to the art, have been achieved by providing a method of treating a mammal with hyperproliferative disease stimulated by a ligand of a member of the epidermal growth factor family of receptors, said method comprising administering to said mammal an effective amount of an antibody or a defective receptor that is an antagonist of a member of the EGF family of receptors.

In another embodiment, the method of the present invention includes treating the mammal with a combination of an effective amount of an EGFR antagonist and a chemotherapeutic agent.

In another embodiment, the method of the present invention includes treating the mammal with a combination of an effective amount of an EGFR antagonist and phototherapy.

In yet another embodiment, the method of the present invention includes treating the mammal with a combination of an effective amount of an EGFR antagonist, phototherapy, and chemotherapy.

### DETAILED DESCRIPTION OF THE INVENTION

The present invention provides an improved method for treating hyperproliferative disease in mammals. The method includes treating the mammal with certain antagonists of the EGF family of receptors (EGFR). In this specification, the term “EGFR” refers to any member of the EGF family of receptors. The EGF family of receptors includes EGFR/HER1. Other members of the EGF family of receptors include HER2, HER3, and HER4.

For the purposes of this specification, “hyperproliferative disease” is defined as a condition caused by excessive growth of non-cancer cells that express a member of the EGFR family of receptors. The excess cells generated by a hyperproliferative disease express EGFR at normal levels or they may overexpress EGFR.

The types of hyperproliferative diseases that can be treated in accordance with the invention are any hyperproliferative diseases that are stimulated by a ligand of EGFR or mutations of such ligands. Some examples of ligands that stimulate EGFR include EGF, TGF-alpha, heparin-binding growth factor (HBGF), β-cellulin, and Cripto-1.

Some examples of hyperproliferative disease include psoriasis, actinic keratoses, and seborrheic keratoses, warts, keloid scars, and eczema. Also included are hyperproliferative diseases caused by virus infections, such as papilloma virus infection. For example, psoriasis comes in many different variations and degrees of severity. Different types of psoriasis display characteristics such a pus-like blisters (pustular psoriasis), severe sloughing of the skin (erythrodermic psoriasis), drop-like dots (guttae psoriasis) and smooth inflamed lesions (inverse psoriasis). The treatment of all types of psoriasis (e.g., psoriasis vulgaris, psoriasis pustulosa, psoriasis erythrodermica, psoriasis arthropathica, parapsoriasis, palmoplantar pustulosis) is contemplated by the invention.

EGFR Antagonists

For the purposes of this specification, an EGFR antagonist is any molecule that inhibits the stimulation of EGFR by an EGFR ligand. Inhibition of stimulation may occur by any mechanism. For example, inhibitors of stimulation include molecules that block the binding of an EGFR and its ligand. Such inhibitors may bind to the EGF receptor or to the EGFR ligand. The inhibition of EGFR stimulation, in turn, inhibits the growth of cells that express EGFR. The growth of excess proliferating cells is sufficiently inhibited in the mammal to prevent or reduce the progression of the hyperproliferative disease.

No particular mechanism of inhibition is implied as operating in the present invention. Nevertheless, EGFR tyrosine kinases are generally activated by means of phosphorylation events. The inhibitors of the present invention may operate by blocking such phosphorylation. Accordingly, phosphorylation assays are useful in predicting the antagonists useful in the present invention. Some useful assays for EGFR tyrosine kinase activity are described in Panek et al., Journal of Pharmacology and Experimental Therapeutics 283: 1433-1444 (1997) and in Batley et al., Life Sciences 62: 143-150 (1998). The description of these assays is incorporated herein by reference.

EGFR antagonists include biological molecules. Biological molecules include all lipids and polymers of monosaccharides, amino acids and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides and proteins. Derivatives of biological molecules further include lipid derivatives of oligosaccharides and polysaccharides, e.g. lipopolysaccharides.

Most typically, biological molecules are antibodies, or functional equivalents of antibodies. Functional equivalents of antibodies have binding characteristics comparable to those of antibodies, and inhibit the growth of cells that express EGFR. Such functional equivalents include, for example, chimerized, humanized and single chain antibodies as well as fragments thereof.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g. the framework variable region, and the constant region of a humanized antibody are those of a human antibody.

For the purposes of this application, suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates. Mice that express human antibodies are preferred. An example of such mice is the XenoMouse™ (Abgenix, Freemont, Calif.) described by Green, L L, “Antibody Engineering Via Genetic Engineering of the Mouse: XenoMouse Strains Are a Vehicle for the Facile Generation of Therapeutic Human Monoclonal Antibodies,”J. Immunol. Methods,” 10; 231(1-2):11-23 (1999).

Functional equivalents of antibodies further include fragments that have binding characteristics that are the same as, or are comparable to, those of the whole antibody. Suitable fragments of the antibody include any fragment that comprises a sufficient portion of the hypervariable (i.e. complementarity determining) region to bind specifically, and with sufficient affinity, to EGFR tyrosine kinase to inhibit growth of cells that express such receptors.

Such fragments may, for example, contain one or both Fab fragments or the F(ab′)₂ fragment. Preferably the antibody fragments contain all six complementarity determining regions of the whole antibody, although functional fragments containing fewer than all of such regions, such as three, four or five CDRs, are also included.

The preferred fragments are single chain antibodies, or Fv fragments. Single chain antibodies are polypeptides that comprise at least the variable region of the heavy chain of the antibody and the variable region of the light chain, with or without an interconnecting linker. Thus, Fv fragment comprises the entire antibody combining site. These chains may be produced in bacteria or in eukaryotic cells.

The antibodies and functional equivalents may be members of any class of immunoglobulins, such as: IgG, IgM, IgA, IgD, or IgE, and the subclasses thereof. The preferred antibodies are members of the IgG1 subclass. The functional equivalents may also be equivalents of combinations of any of the above classes and subclasses.

Antibodies may be made from the desired receptor by methods that are well known in the art. The receptors are either commercially available, or can be isolated by well-known methods. For example, methods for isolating and purifying EGFR are found in Spada, U.S. Pat. No. 5,646,153 starting at column 41, line 55. The method for isolating and purifying EGFR described in the Spada patent is incorporated herein by reference.

Methods for making monoclonal antibodies include, for example, the immunological method described by Kohler and Milstein in Nature 256:495-497 (1975) and by Campbell in “Monoclonal Antibody Technology, The Production and Characterization of Rodent and Human Hybridomas” in Burdon et al. Eds, Laboratory Techniques in Biochemistry and Molecular Biology, Volume 13, Elsevier Science Publishers, Amsterdam (1985). The recombinant DNA method described by Huse et al. in Science 246:1275-1281 (1989) is also suitable.

Briefly, in order to produce monoclonal antibodies, a host mammal is inoculated with a receptor or a fragment of a receptor, as described above, and then, optionally, boosted. In order to be useful, the receptor fragment must contain sufficient amino acid residues to define the epitope of the molecule being detected. If the fragment is too short to be immunogenic, it may be conjugated to a carrier molecule. Some suitable carrier molecules include keyhold limpet hemocyanin and bovine serum albumin. Conjugation may be carried out by methods known in the art. One such method is to combine a cysteine residue of the fragment with a cysteine residue on the carrier molecule.

Spleens are collected from the inoculated mammals a few days after the final boost. Cell suspensions from the spleens are fused with a tumor cell. The resulting hybridoma cells that express the antibodies are isolated, grown, and maintained in culture.

Suitable monoclonal antibodies as well as growth factor receptor tyrosine kinases for making them are also available from commercial sources, for example, from Upstate Biotechnology, Santa Cruz Biotechnology of Santa Cruz, Calif., Transduction Laboratories of Lexington, Ky., R&D Systems Inc of Minneapolis, Minn., and Dako Corporation of Carpinteria, Calif.

Methods for making chimeric and humanized antibodies are also known in the art. For example, methods for making chimeric antibodies include those described in U.S. patents by Boss (Celltech) and by Cabilly (Genentech). See U.S. Pat. Nos. 4,816,397 and 4,816,567, respectively. Methods for making humanized antibodies are described, for example, in Winter, U.S. Pat. No. 5,225,539.

Antibodies or antibody fragments can also be isolated from antibody phage libraries generated using techniques, for example, described in McCafferty et al., Nature, 348: 552-554 (1990), using the antigen of interest to select for a suitable antibody or antibody fragment. Clackson et al., Nature, 352: 624-628 (1991.) and Marks et al., J. Mol. Biol., 222: 581-597 (1991) describe the isolation of murine and human antibodies, respectively, using phage libraries, Subsequent publications describe the production of high affinity (mM range) human antibodies by chain shuffling (Mark et al., Bio/Technol. 10; 779-783 (1992), as well as combinatorial infection and in vivo recombination as a strategy for constructing very large phage libraries (Waterhouse et al., Nuc. Acids Res., 21: 2265-2266 (1993). These techniques are viable alternatives to traditional monoclonal antibody hybridoma techniques for isolation of “monoclonal” antibodies (especially human antibodies).

The preferred method for the humanization of antibodies is called CDR-grafting. In CDR-grafting, the regions of the mouse antibody that are directly involved in binding to antigen, the complementarity determining region or CDRs, are grafted into human variable regions to create “reshaped human” variable regions. These fully humanized variable regions are then joined to human constant regions to create complete “fully humanized” antibodies.

In order to create fully humanized antibodies that bind well to an antigen, it is advantageous to design the reshaped human variable regions carefully. The human variable regions into which the CDRs will be grafted should be carefully selected, and it is usually necessary to make a few amino acid changes at critical positions within the framework regions (FRs) of the human variable regions.

For example, the reshaped human variable regions may include up to ten amino acid changes in the FRs of the selected human light chain variable region, and as many as twelve amino acid changes in the FRs of the selected human heavy chain variable region. The DNA sequences coding for these reshaped human heavy and light chain variable region genes are joined to DNA sequences coding for the human heavy and light chain constant region genes, preferably γ1 and κ, respectively. The reshaped humanized antibody is then expressed in mammalian cells and its affinity for its target compared with that of the corresponding murine antibody and chimeric antibody.

Methods for selecting the residues of the humanized antibody to be substituted and for making the substitutions are well known in the art. See, for example, Co et al., Nature 351:501-502 (1992); Queen et al., Proc. Natl. Acad. Sci. 86: 10029-1003 (1989) and Rodrigues et al., Int. J Cancer Supplement 7: 45-50 (1992). A method for humanizing and reshaping the 225 anti-EGFR monoclonal antibody described by Goldstein et al. in PCT application WO 96/40210. This method can be adapted to humanizing and reshaping antibodies against other growth factor receptor tyrosine kinases.

Other methods for making single chain antibodies are also known in the art. Such methods include screening phage libraries transfected with immunoglobulin genes described in U.S. Pat. No. 5,565,332; U.S. Pat. No. 5,5837,242; U.S. Pat. No. 5,855,885; U.S. Pat. No. 5,885,793; and U.S. Pat. No. 5,969,108. Another method includes the use of a computer-based system for designing linker peptides for converting two separate polypeptide chains into a single chain antibody described in U.S. Pat. No. 4,946,778; U.S. Pat. No. 5,260,203; U.S. Pat. No. 5,455,030; and U.S. Pat. No. 5,518,889.

Other methods for producing the functional equivalents described above are disclosed by Wels et al. in European patent application 502 812 and Int. J Cancer 60:137-144 (1995); PCT Application WO 93/21319; European Patent Application 239 400, PCT Application WO 89/09622; European Patent Application 338 745; U.S. Pat. No. 5,658,570; U.S. Pat. No. 5,693,780; and European Patent Application EP 332 424.

Preferred EGFR antibodies are the chimerized, humanized, and single chain antibodies derived from a murine antibody called 225, which is described in U.S. Pat. No. 4,943,533. The patent is assigned to the University of California and licensed exclusively to ImClone Systems Incorporated. The 225 antibody is able to inhibit the growth of cultured EGFR/HER1-expressing tumor cells in vitro as well as in vivo when grown as xenografts in nude mice. See Masui et al., Cancer Res., 44:5592-5598 (1986).

In one example of the present invention, a human patient with psoriasis was treated with a chimerized version of the 225 antibody described above. As can be seen in Example 2 below, the C225 antibody was effective in treating the psoriasis.

The chimerized, humanized, and single chain antibodies described above may be derived from murine antibody 225 can be made from the 225 antibody, which is available from the ATCC. Alternatively, the various fragments needed to prepare the chimerized, humanized, and single chain 225 antibodies can be synthesized from the sequence provided in Wels et al. in Int. J. Cancer, 60:1.37-144 (1995). The chimerized 225 antibody (c225) can be made in accordance with the methods described above. Humanized 225 antibody can be prepared in accordance with the method described in example IV of PCT application WO 96/40210, which is incorporated herein by reference. Single chain 225 antibodies (Fv225) can be made in accordance with methods described by Wels et al. in Int. J Cancer, 60:137-144 (1995) and in European patent application 502 812.

The sequences of the hypervariable (CDR) regions of the light and heavy chain of the 225 antibody are reproduced below. The amino acid sequence is indicated below the nucleotide sequence.

| | |
|---|---|
| HEAVY CHAIN HYPERVARIABLE REGIONS (VH): | |
| CDR1 | |
| AACTATGGTGTACAC | (SEQ ID 1) |
| N Y G V H | (SEQ ID 2) |
| CDR2 | |
| GTGATATGGAGTGGTGGAAACACAGACTATAATACACCTTTCACATCC | (SEQ ID 3) |
| V I W S G G N T D Y N T P F T S | (SEQ ID 4) |
| CDR3 | |
| GCCCTCACCTACTATGATTACGAGTTTGCTTAC | (SEQ ID 5) |
| A L T Y Y D Y E F A Y | (SEQ ID 6) |
| LIGHT CHAIN HYPERVARIABLE REGIONS (VL): | |
| CDR1 | |
| AGGGCCAGTCAGAGTATTGGCACAAACATACAC | (SEQ ID 7) |
| R A S Q S I G T N I H | (SEQ ID 8) |
| CDR2 | |
| GCTTCTGAGTCTATCTCT | (SEQ ID 9) |
| A S E S I S | (SEQ ID 10) |
| CDR3 | |
| CAACAAAATAATAACTGGCCAACCACG | (SEQ ID 11) |
| Q Q N N N W P T T | (SEQ ID 12) |

Biological molecules useful as antagonists also include defective receptors capable of binding to an EGF receptor ligand, but incapable of transducing a signal to the cell. For example, the catalytic domain itself can be defective. Alternatively, the defective receptors may be soluble receptors of the EGFR family. Soluble receptors lack a catalytic domain and, optionally, a transmembrane domain. The preferred soluble receptors are soluble EGFR/HER1 receptors. (Exp. Cell Res., 241(1):161-170; Proc. Natl. Acad. Sci., 92(23):10457-61).

In addition to the biological molecules discussed above, the antagonists useful in the present invention may also be small molecules. Any molecule that is not a biological molecule is considered in his specification to be a small molecule. Although small molecule EGFR antagonists have been already suggested for use in treating psoriasis, they have not been suggested for use in treating hyperproliferative disease in combination with phototherapy and/or chemotherapy described below.

Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides, and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds.

Examples of such small molecules include the tyrphostins described by Dvir, et al., J. Cell Biol., 113:857-865 (1991); the quinazoline compounds described in U.S. Pat. No. 6,004,967; the styryl substituted heteroaryl compounds disclosed in U.S. Pat. No. 5,656,655; the bis mono and/or bicyclic aryl, heteroaryl, carbocyclic, and heterocarbocyclic compounds disclosed in U.S. Pat. No. 5,646,153; the tricyclic pyrimidine compounds disclosed in U.S. Pat. No. 5,679,683; or the heteroarylethenediyl compounds disclosed in U.S. Pat. No. 5,196,446.

### Administration of EGFR Antagonists

The present invention includes administering an effective amount of the EGFR antagonist to a mammal, preferably a human patient. The EGFR antagonist may be administered topically, for example, in a cream or emollient; or systemically, for example, by the parenteral and enteral routes.

For example, EGFR antagonists utilized in the present invention can easily be administered intravenously (e.g., intravenous injection) which is a preferred route of delivery. Intravenous administration can be accomplished by combining the EGFR antagonists with a suitable pharmaceutical carrier (vehicle) or excipient, as understood by those skilled in the art. The EGFR antagonist may be administered with adjuvants, such as, for example, BCG, immune system stimulators and chemotherapeutic agents.

The EGFR antagonists of the present invention significantly inhibit the excess growth of cells associated with hyperproliferative disease when administered to a mammal in an effective amount. As used herein, an effective amount is that amount effective to achieve the specified result of inhibiting the growth of such excess cells.

Optimal doses of EGFR antagonists can be determined by physicians based on a number of parameters including, for example, age, sex, weight, severity of the condition being treated, the compound being administered, and the route of administration. In general, a serum concentration of antagonists that permits saturation of the target receptor is desirable. For example, a concentration of antibodies, functional equivalents of antibodies, and/or defective receptors in excess of approximately 0.1 nM is normally sufficient. A dose of 100 mg/m² of C225 generally provides a serum concentration of approximately 20 nM for approximately eight days.

As a rough guideline, doses of antibodies may be given weekly in amounts of 10-300 mg/m². Equivalent doses of antibody fragments should be used at more frequent intervals in order to maintain a serum level in excess of the concentration that permits saturation of the receptors.

### Combination Therapy

Hyperproliferative disease can be treated with an effective amount of an EGFR antagonist in combination with any conventional treatment. Examples of conventional treatment include chemotherapeutic agents, phototherapy or combinations thereof.

Thus, in one embodiment of the invention, hyperproliferative disease is treated by administering both an EGFR antagonist and a chemotherapeutic agent. As an example, when the hyperproliferative disease is psoriasis, there are a variety of conventional chemotherapeutic agents available. The chemotherapeutic agents generally fall into two categories; systemic and topical.

Topical chemotherapeutic agents for psoriasis include anthralin, calcipotriene, coal tar, corticosteroids, emollients, keratolytics, and tazarotene. Topical steroids are one of the most common therapies prescribed for mild to moderate psoriasis. Topical steroids are applied to the surface of the skin, but some are injected into the psoriasis lesions. Coal tar is a very old remedy which is sold both over-the-counter and in prescription form. Anthralin is a prescription compound that has been used to treat psoriasis for over a hundred years. Calcipotriene is a synthetic vitamin D3 analog, usually used to treat mild to moderate side effects. The Food and Drug Administration approved tazarotene (brand name TAZORACc™) in mid-1997. Retinoids are a family of drugs related to vitamin A. Keratolytics, such as salicylic acid, are sometimes used to remove scaling caused by psoriasis. Emollients are usually only helpful in keeping the skin pliable, but typically do not address the underlying condition.

Systemic chemotherapeutic agents for psoriasis include antibiotics, antimicrobials, cyclosporine, methotrexate, and oral retinoids, such as acitretin, etretinate, and isotretinoin. Cyclosporin in a new oral drug made by Novartis Pharmaceuticals Corporation and is sold under the brand name NEORAL™. It is typically used to treat adults who have severe plaque psoriasis. Cyclosporin inhibits immune activity. Such inhibition appears to slow the abnormally rapid skin cell turnover and reduce the number of activated inflammatory cells in the skin. Methotrexate is an internal medication that can be given either as a pill or as an injection for psoriasis. Methotrexate has been found to be effective in treating some types of psoriasis, but careful monitoring is needed to avoid side effects such as nausea, fatigue, loss of appetite and mouth sores and, in more extreme cases, liver damage. The retinoid family of drugs is related to vitamin A. Before 1998, TEGISON™ was a retinoid commonly used to treat severe psoriasis. This drug has since been phased out in favor of SORIATANE™. ACCUTANE™, the brand name of the prescription medication isotretinoin, is a retinoid drug more typically used for cystic acne and is less effective than SORIATANE™. Other systemic treatments of psoriasis include hydroxyurea, NSAIDS, sulfasalazine, and 6-thioguanine. Antibiotics and antimicrobials can be used to treat or prevent infection that can cause psoriasis to flare and worsen.

The EGFR antagonist can also be combined with chemotherapeutic drugs used to inhibit the proliferation of cells, but not conventionally utilized in treating psoriasis. For example, the method of the invention can include the administration of a chemotherapeutic drug conventionally used to combat cancer. Examples of such anticancer chemotherapeutic drugs include amifostine, (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar), daunorubicin, daunorubicin lipo (daunoxome), procarbazine, milomycin, cytarabine, etoposide, methotrexate, 5-fluorouracil, vinblastine, vincristine bleomycin, paclitaxel (taxol), docetaxel (taxotere), aldesleukin, asparaginase, busulfan, carboplatin, cladribine, camptothecin, CPT-11 (irinotecan), 10-hydroxy-7-ethyl-camptothecin (SN38), dacarbazine, floxuridine, fludarabine, hydroxyurea, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof. Cisplatin is preferred.

In another embodiment of the invention, hyperproliferative disease is treated with an effective amount of an EGFR antagonist in combination with phototherapy. Phototherapy is the administration of any wavelength of light that is at least partially effective in reducing the symptoms of hyperproliferative disease, such as psoriasis. Examples include ultraviolet A (UVA) and ultraviolet B (UVB). Medically supervised administration of UVB has been used to control widespread or localized areas of stubborn and unmanageable psoriasis lesions. Natural sunlight has also been demonstrated to help the symptoms of psoriasis in certain cases.

The phototherapy is administered in accordance with well known standard techniques with standard equipment manufactured for this purpose. The dose of phototherapy depends on numerous factors as is well known in the art. Such factors include the organ being treated, the healthy organs in the path of the phototherapy that might inadvertently be adversely affected, the tolerance of the patient for phototherapy, and the area of the body in need of treatment. It should be emphasized, however, that the invention is not limited to any particular dose. The dose will be determined by the treating physician in accordance with the particular factors in a given situation, including the factors mentioned above.

In another embodiment the invention, the hyperproliferative disease is treated with an effective amount of an EGFR antagonist in combination with both a chemotherapeutic agent and phototherapy. The combination of a chemtotherapeutic agent and phototherapy is often called photochemotherapy. When using photochemotherapy in the method of the invention, the medical provider can vary the treatments and/or dosage to determine the most effective regimen.

One example of photochemotherapy for use against psoriasis is PUVA, which is an acronym for the combination of the drug psoralen with ultraviolet light A. PUVA treatments are medically supervised and there are a variety of methods to deliver the therapy. The most common method is to administer the psoralen orally and then administer UVA to affected areas.

Sometimes rotational therapy is used in which various therapies, such as chemotherapy and phototherapy, are used consecutively. For example, day treatment programs are available for people with widespread psoriasis. Patients typically spend six to eight hours every day for two to four weeks in the day treatment program where they are treated with tar, anthralin and UVB. Special centers have been created in certain metropolitan areas for such treatment.

The method of the invention contemplates the use of an EGFR antagonist, in any combination with chemotherapy or phototherapy, or any other treatments effective for treating a hyperproliferative disease.

In a preferred embodiment, there is synergy with the EGFR antagonist and conventional treatment, such as chemotherapeutic agents or phototherapy or combinations thereof. In other words, the inhibition of excess cell growth by the EGFR antagonist is enhanced when combined with chemotherapeutic agents or phototherapy or combinations thereof. Synergy may be shown, for example, by greater inhibition of excess cell growth with combined treatment than would be expected from treatment with either the EGFR antagonist, chemotherapeutic agent, or phototherapy alone. Preferably, synergy is demonstrated by complete clearing of the symptoms of hyperproliferative disease, where clearing of the symptoms is not obtainable from treatment with EGFR antagonist, chemotherapeutic agent or phototherapy alone.

The EGFR antagonist can be administered before, during, or after commencing chemotherapeutic agent or phototherapy therapy, as well as any combination thereof, i.e. before and during, before and after, during and after, or before, during, and after commencing the chemotherapeutic agent and/or phototherapy.

### EXAMPLE 1

### Protocol

Human patients suffering from psoriasis are treated with a combination of an EGFR/HER1 antagonist (chimeric anti-EGFR monoclonal antibody, C225) and cisplatin. The patients receive weekly infusions of C225 at loading/maintenance doses of 100/100, 400/250, or 500/250 mg/m² in combination with 100 mg/m² of cisplatin every three weeks. Samples are obtained at baseline, 24 hours after the initial infusion and 24 hours before the third infusion to assess EGFR saturation and function. EGFR saturation is assessed by immunohistochemistry (IHC) using M225 (murine counterpart of C225) as primary antibody and antimouse IgG as secondary antibody to detect unoccupied EGFR. The EGFR function is assessed by IHC using an antibody specific for activated EGFR (Transduction Labs) and measurement of EGFR tyrosine kinase activity on lysates after clearing the C225-EGFR complexes. A dose dependent increase in receptor saturation is noted with greater than 70% receptor saturation through 500/250 mg/m² dose levels. Similarly, a significant reduction of EGFR-tyrosine kinase activity is noted with no detectable activity in 67% of the patients at doses of 100/100 mg/m², suggesting functional saturation.

### EXAMPLE 2

### Clinical Trial

In a clinical trial, one human patient with psoriasis and refractory colon cancer was treated with a combination of an EGFR/HER1 antagonist (chimeric anti-EGFR monoclonal antibody, C225) and CPT-11 (irinotecan). The patient received weekly infusions of C225 at a loading dose of 400 mg/m² in combination with 125 mg/m² of CPT-11. Maintenance doses of 250 mg/m² C225 in combination with 69-125 mg/m² of CPT-11 were administered on a weekly basis. Clinically, the patient had a complete response with respect to psoriasis. The dosing schedule is summarized in Table 1 below.

TABLE 1
Clinical Trial

| C225/CPT-11 | | C225 | |
|---|---|---|---|
| Weekly dose in | C225/CPT-11 | Infusion Time | CPT-11 Infusion |
| mg/m² | (Actual dose in mg) | (minutes) | Time (minutes) |
| 400/125 | 576/180 | 120 | 90 |
| 250/125 | 360/180 | 60 | 90 |
| 250/CPT-11 | 360/0 | 60 | N/A |
| Held | | | |
| 250/94 | 360/135 | 50 | 75 |
| 250/69 | 360/100 | 60 | 85 |
| 250/69 | 360/100 | 60 | 75 |

What is claimed is:

1. A method of treating a human with psoriasis, comprising systemically administering to said human an amount of an EGFR/HER1 antibody effective to treat psoriasis.

2. A method according to claim 1 wherein the antibody is a monoclonal antibody specific for EGFR/HER1 or a fragment that comprises the hypervariable region thereof.

3. A method according to claim 2 wherein the monoclonal antibody is chimerized or humanized.

4. A method according to claim 2 wherein the monoclonal antibody inhibits EGFR/HER1 phosphorylation.

5. A method according to claim 1 wherein the antibody is administered intravenously.

6. A method according to claim 1 wherein the antibody comprises the following CDRs having the following amino acid sequences:
CDR1, SEQ ID 2;
CDR2, SEQ ID 4;
CDR3, SEQ ID 6;
CDR4, SEQ ID 8;
CDR5, SEQ ID 10;
CDR6, SEQ ID 12.

7. A method according to claim 6 wherein the antibody is 225 or chimeric 225.


SEQUENCE LISTING (12 sequences)

SEQ ID NO: 1 - DNA, 16 bp, Mouse
aactatggtgtaacac

SEQ ID NO: 2 - protein, 5 aa, Mouse
NYGVH

SEQ ID NO: 3 - DNA, 48 bp, Mouse
gtgatatggagtggtggaaacacagactataatacacctttcacatcc

SEQ ID NO: 4 - protein, 15 aa, Mouse
VIWSGGNTDYNTPFT

SEQ ID NO: 5 - DNA, 33 bp, Mouse
gccctcacctactatgattacgagtttgcttac

SEQ ID NO: 6 - protein, 11 aa, Mouse
ALTYYDYEFAY

SEQ ID NO: 7 - DNA, 33 bp, Mouse
agggccagtcagagtattggcacaaacatacac

SEQ ID NO: 8 - protein, 11 aa, Mouse
RASQSIGTNIH

SEQ ID NO: 9 - DNA, 18 bp, Mouse
gcttctgagtctatctct

SEQ ID NO: 10 - protein, 6 aa, Mouse
ASESIS

SEQ ID NO: 11 - DNA, 27 bp, Mouse
caacaaaataataactggccaaccacg

SEQ ID NO: 12 - protein, 9 aa, Mouse
QQNNNWPTT